(19) United States
(12) Patent Application Publication
(10) Pub. No.: US 2015/0004139 A1
(43) Pub. Date: Jan. 1, 2015

(54) Emollient Compounds Useful for Treating Dry Skin Conditions

(71) Applicants: Rodney David Lewis, Joplin, MO (US); Paige Mashara Lewis, Joplin, MO (US)

(72) Inventors: Rodney David Lewis, Joplin, MO (US); Paige Mashara Lewis, Joplin, MO (US)

(21) Appl. No.: 14/222,682

(22) Filed: Jun. 16, 2014

Related U.S. Application Data
(60) Provisional application No. 61/804,880, filed on Mar. 25, 2013.

(51) Int. Cl. A61K 8/97; A61K 8/365; A61Q 19/00; A61K 8/36

(52) U.S. Cl. CPC A61K 8/97; A61K 8/361; A61K 8/365; A61Q 19/007; A61K 2800/70
USPC 424/93.2; 424/727; 424/731; 424/735; 424/751; 424/757

(57) ABSTRACT

The present invention provides a composition and method useful for creating a class of compounds that provide an emollient effect to the skin. The base formulation includes mixtures of certain oils that have high concentrations of Linoleic Acid, an essential fatty acid. Other oils are chosen which have high concentrations of Gamma-Linolenic Acid, Ricinoleic Acid and Oleic Acid. We have found that the incorporation of dl-alpha-tocopheryl and dl-alpha-tocopherol (vitamin E) improves the formulation and provides an anti-oxidative effect which preserves the end product and provides for a longer shelf life. The method disclosed creates free fatty acids, utilizing base catalyzed trans-esterification, inter-esterification and intra-esterification. The final products are lipid compounds which aid in treating dry skin conditions caused by deficiencies in Linoleic Acid or other fatty acids.

### CROSS-REFERENCE TO RELATED APPLICATIONS

This application claims priority to U.S. Provisional Patent Application Ser. No. 61/804,880 filed on Mar. 25, 2013 to Rodney David Lewis and Paige Mashara Lewis of Joplin, Mo. entitled “Emollient Compounds Useful for Treating Dry Skin Conditions,” currently pending, the entire disclosure is incorporated herein by reference.

### BACKGROUND OF THE INVENTION

The vast majority of skin creams and emollients in the marketplace today are mostly water based compounds that contain ingredients that claim to moisturize and protect the skin. These products may contain mixtures of synthetic, plant, animal or petroleum derived ingredients, including: petrolatum, silicone, mineral oil, glycerin, fruit and nut extracts, aloe, emu oil, lanolin, and homogenizing agents.

A common complaint about these products is that their effects are temporal. Once applied, their efficacy typically lasts for only a few hours, at best. For persons who suffer from dry skin conditions, especially those caused by deficiencies in the essential fatty acids (EFA) Linoleic Acid (LA) and Gamma Linolenic Acid (GLA), there is little and no lasting relief. None of the compounds in the market today alleviate the potential deficiency of these fatty acids.

Healthy human cells transform LA into GLA by the enzymatic action of delta-6-desaturase, but there may be inhibitors that interfere with this enzyme, including excess cholesterol, excess saturated and monounsaturated long-chain fatty acids (a major constituent in Western diets), trans-fats, alcohol, aging, zinc deficiency, and high sugar consumption. By providing GLA directly, we can bypass the deficiency or blocked enzyme, according to the book: Fats that Heal, Fats that Kill by Udo Erasmus (Alive Books Burnaby BC Canada. 1996. Pages 270-271).

According to Drs. Alex Richardson and Marion Ross at the University Lab of Physiology at Oxford, people whose diets are deficient in certain polyunsaturated fatty acids, like LA and GLA, may have these common symptoms: scaly dermatitis, skin lesions, atopic eczema, rough or dry chapped skin, and follicular keratosis. Indeed, according to the monograph, “Essential Fatty Acids and Skin Health,” by Giana Angelo, Ph.D., at the Linus Pauling Institute at Oregon State University, these polyunsaturated fatty acids (PUFA) “play a critical role in normal skin function and appearance” and “essential fatty acid deficiency (EFAD) significantly affects skin function and appearance. EFAD is characterized by hyperproliferation of the epidermis, dermatitis, and increased transepidermal water loss (TEWL). TEWL reflects the integrity of the barrier function of the skin and is directly related to the EFA composition of structural lipids in the stratum corneum.”

The problem with the state of the art in moisturizers and skin lotions is that most of these compounds aim to moisturize by adding water to the skin. Indeed, most all have water as their primary constituent. Normal skin, however, functions as a barrier; moisture is normally repelled by the skin. Any moisture present in most skin creams and lotions rapidly evaporates. The homogenizing agents or surfactants that are left on the skin surface can then re-attach themselves to skin oils and soapy water (when one washes their hands) and strip the natural oils away from the skin. Thus these lotions may contribute to the very problem they aim to solve.

Normal skin doesn't need topically applied water for moisturizing. The water in skin cells is there as a result of normal physiological processes. What is needed is to keep this water within the cell, namely an improvement in barrier function. A most critical skin component that is responsible for its barrier function is intercellular lipids: “Lipid layers hold water and surround corneocytes to provide permeability barrier. The intercellular lipids and corneocytes containing proteins and natural moisturizing factors work together to provide an efficient barrier against water loss and water retention to maintain the flexibility of the skin. The protective forces shield the skin from desiccation and environmental assaults.” [Skin Physiology, Irritants, Dry Skin and Moisturizers by Christina Marino, MD, MPH. Report Number 56-2-2001a (revised June 2006)].

It is well known in the art that the loss of moisture within the skin is called transepidermal water loss (TEWL) and is the major factor responsible for dry skin. Most moisturizers use ingredients that are either humectants or occlusive. The humectants attract water to the skin. Examples include propylene glycol, glycerin and urea. The occlusive agents work by physically blocking water loss and include: petrolatum, lanolin, beeswax, and oils. There are also “barrier repairing” moisturizers that contain lipids similar to the intercellular lipids of the skin, like cholesterol, ceramide, and fatty acids. [Skin Physiology, Irritants, Dry Skin and Moisturizers by Christina Marino, MD, MPH. Report Number 56-2-2001a (revised June 2006)].

U.S. Pat. No. 4,389,418 discloses the use of a cationic surfactant, mineral oil and petrolatum in a water emulsion to assist in forming an occlusive water-repellant layer on the skin.

U.S. Pat. No. 6,264,963 discloses a humectant system comprising of glycerin and plant-derived oils, petrolatum or mineral oil, surfactants, sodium pyrrolidone carboxylate, beta-glucan, lactic acid, and lactic acid salts, formulated in a water emulsion.

A need exists for compounds that are capable of limiting TEWL while also providing the essential fatty acids needed for proper skin function and appearance. The lipid nature of this invention, in addition to providing free PUFAs and the essential LA, also promotes “barrier repairing” activity by the skin as the compounds rapidly absorb into the skin and are incorporated into the dermis and epidermis. Indeed, in her research paper, Dr. Giana Angelo affirms: “topical application is also a successful route of EFA delivery to the skin. Symptoms of EFA deficiency (EFAD) in both animals and humans can be reversed by either topical application or ingestion of oils rich in LA.” [“Essential Fatty Acids and Skin Health” by Giana Angelo, Ph.D.; Linus Pauling Institute, Oregon State University].

### BRIEF SUMMARY OF THE INVENTION

This invention relates to new skin conditioning emollient crémes that contains free essential fatty acids needed for healthy skin. In particular, the invention relates to novel lipid emollients that may be derived from natural oils and tocopheryls that contains—in free form—the essential Linoleic Acid, the polyunsaturated fatty acids: Gamma Linolenic Acid, Ricinoleic Acid, and the monosaturated Oleic Acid. Unlike the existing art, the instant invention contains neither humectant nor occlusive ingredients. The compounds described herein are readily absorbed into the skin and restore its barrier function. In independent laboratory RIPT testing these type compounds proved to be non-irritating and safe for human use.

One embodiment of the present invention is directed generally to blends of organic oils, dl-alpha-tocopheryl, dl-alpha-tocopherol, and a free fatty acid, like Stearic Acid, which is dissolved into the oils. This blend is subsequently inter-esterified, intra-esterified and trans-esterified using the fatty acid and a base catalyst. The oils chosen depend on their fatty acid content and have typically high concentrations of Linoleic Acid (LA), Gamma-Linolenic Acid (GLA), or Ricinoleic Acid (RA). For example these oils are good choices: safflower oil, sunflower oil, castor oil, evening primrose oil, borage oil, etc. When these oils are esterified using a suitable base catalyst, like sodium hydroxide, and a fatty acid like Stearic Acid (SA) there are rapid exothermic reactions that take place. Some of the fatty acids locked in the triglyceride structure of the oils are replaced with the SA and are left to continue the esterification process. The hydroxyl moiety of the ricinoleic acid may form esters with other fatty acids. A rearrangement of the fatty acids take place in the mixture until the reactions slow down as evidenced by a temperature decrease in the reactants. Some of the previously triglyceride-bound fatty acids, namely LA and GLA, are present in free salt forms. Some of the oils are also partially saponified, but not to completion. The overall mixture is stirred. Excess water and sodium hydroxide is purged from the compound and is removed. The compound is allowed to rest for a time as the temperature falls to ambient levels; a rest period of 12-24 hours is normal. A second step in the process is to homogenize the secondary product with distilled water so that the entire compound is uniformly distributed in liquid form. This may be accomplished with a high shear mixer. After homogenization the physical state is a fairly viscous and frothy liquid. A deodorization step is optional at this point. A third step in the process is to acidify the resultant compound with a suitable acid solution, like citric acid or ascorbic acid in dissolved in water. The acidification takes place simultaneously with a high shear mixing operation until the pH of the aqueous solution is approximately 6.5-7.0 at which point the acidification is complete and the fatty compounds separate and float on top of the aqueous salt solution. A fourth step involves the physical separation of the fatty compounds from the water using a suitable method, like filtration accomplished with a screen or sieve. Suitable fragrances can be added either toward the end of acidification in the third step or in the fourth step after the lipid créme has been separated from the aqueous solution. The lipid compound can then be vacuum packed in preparation for transport or subsequent filling operations. The novel and final product is a rich créme that is rapidly absorbed by the skin and has an astonishing emollient effect.

Another embodiment of the present invention involves interesterification of a singular oil, where there is no mixture with other oils. The interesterification proceeds to completion. In the second step, mixtures of these singular compounds are homogenized, then acidified in the third step and separated from the aqueous solution in a fourth step.

Another embodiment of the present invention involves utilizing mixtures of both inter-esterified and intra-esterified oils in the second step, followed by acidification in the third step and separation in the fourth step.

Note that in the above discussion we define trans-esterification traditionally, where in the presence of a base catalyst, a free fatty acid, like Stearic Acid, replaces a fatty acid in a triglyceride. We define inter-esterification as that reaction where a liberated fatty acid then replaces another fatty acid from a triglyceride molecule, belonging to the same oil. We define intra-esterification as that reaction where a liberated fatty acid then replaces another fatty acid from a triglyceride molecule belonging to a different oil.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a molecular diagram of Stearic Acid (SA).

FIG. 2 is a molecular diagram of Linoleic Acid (LA).

FIG. 3 is a molecular diagram of Gamma Linolenic Acid (GLA).

FIG. 4 is a molecular diagram of Oleic Acid (OA).

FIG. 5 is a molecular diagram of Ricinoleic Acid (RA).

FIG. 6 shows the structure of a typical triglyceride. In this particular example, there are three fatty acids bound to a glycerol carbon by an ester bond. Two of these fatty acids are identical in this example and one is different. The triglyceride of FIG. 6 contains the two fatty acids: Stearic Acid (FIG. 1) and Oleic Acid (FIG. 4).

FIG. 7 shows another triglyceride. Like FIG. 6, this example shows three fatty acids bound to a glycerol carbon by an ester bond. Two of these fatty acids are identical and one is different. FIG. 7 contains the two fatty acids: Stearic Acid (FIG. 1) and Linoleic Acid (FIG. 2). Linoleic Acid is a polyunsaturated fatty acid (PUFA) that contains more than one carbon-carbon double bond. In FIG. 7 one can see that there are two linoleic acid chains, which have double bonds at positions w-9 and w-12 of each fatty acid. Linoleic Acid has special importance in the instant invention because it is also an essential fatty acid: human bodies require it, but cannot synthesize it. Thus the only way we can obtain this fatty acid is either by ingestion of a food that contains it or topical application.

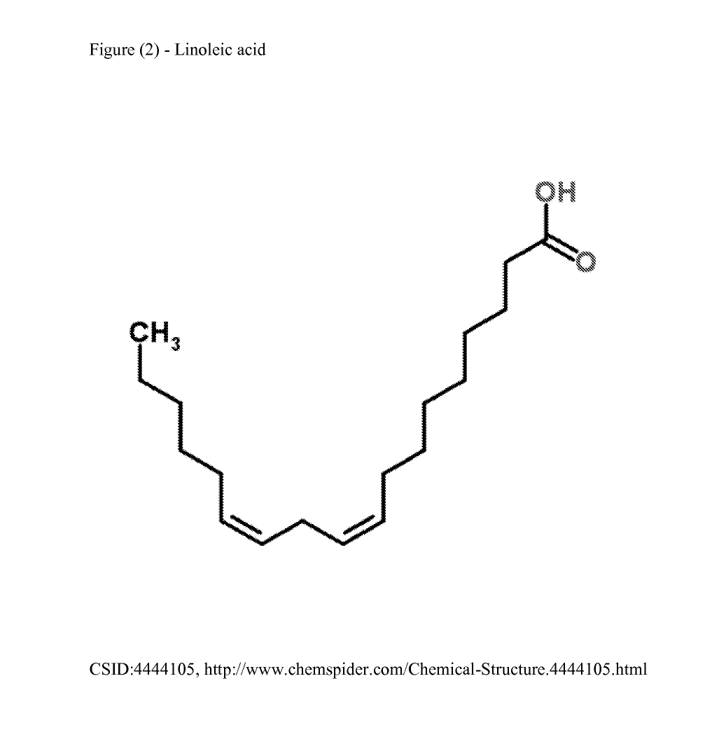

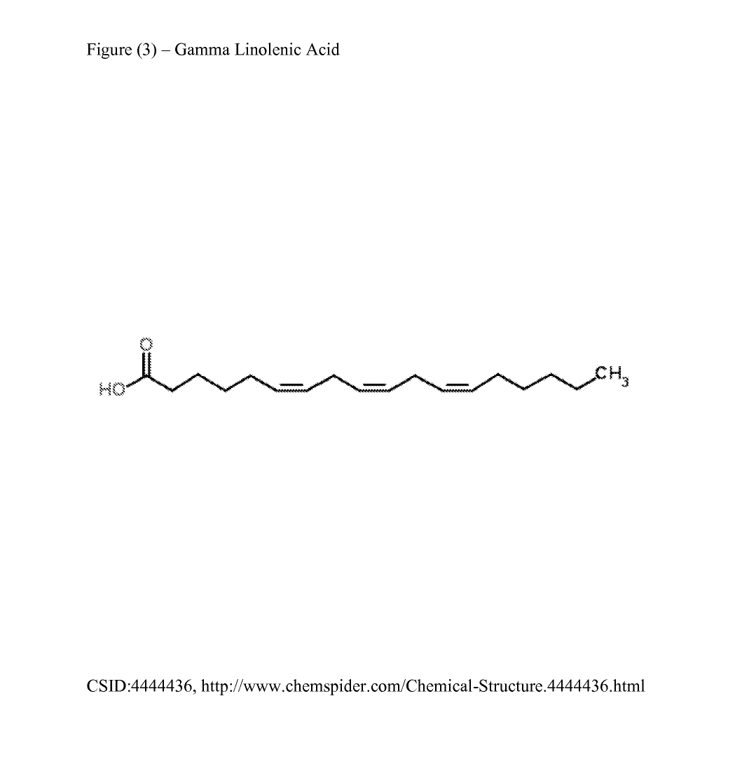

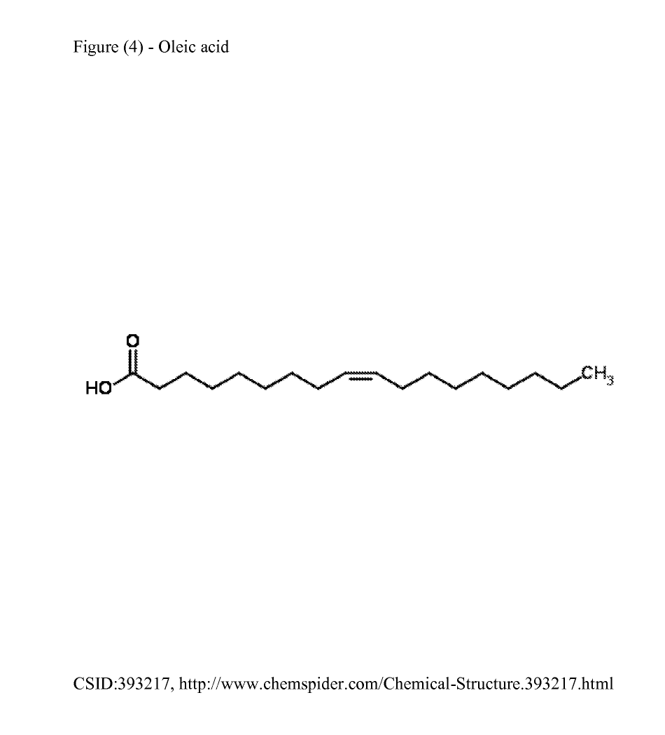

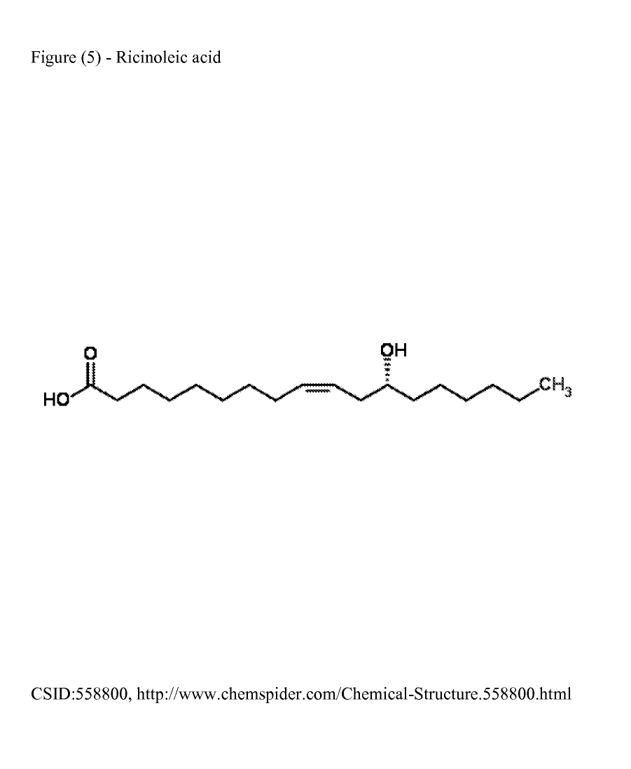

FIG. (8) shows another triglyceride. Again, this example shows three fatty acids bound to a glycerol carbon by an ester bond. Each of these fatty acids is identical: Linoleic Acid (FIG. 2).

FIG. (9) shows another triglyceride. Like the previous figures, FIG. (9) shows three fatty acids bound to a glycerol carbon by an ester bond. Two of these fatty acids are identical and one is different. FIG. (9) contains the two fatty acids: Palmitic Acid and Oleic Acid. Palmitic acid is fully saturated, like Stearic Acid, and contains no carbon-carbon double bonds. There are sixteen carbons which comprise the Palmitic Acid chain.

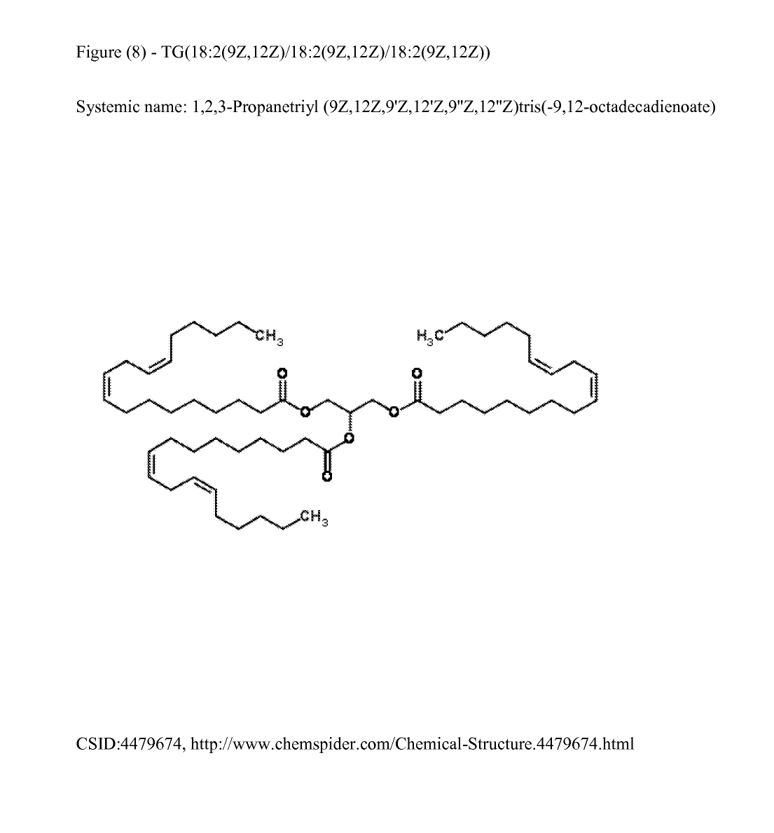

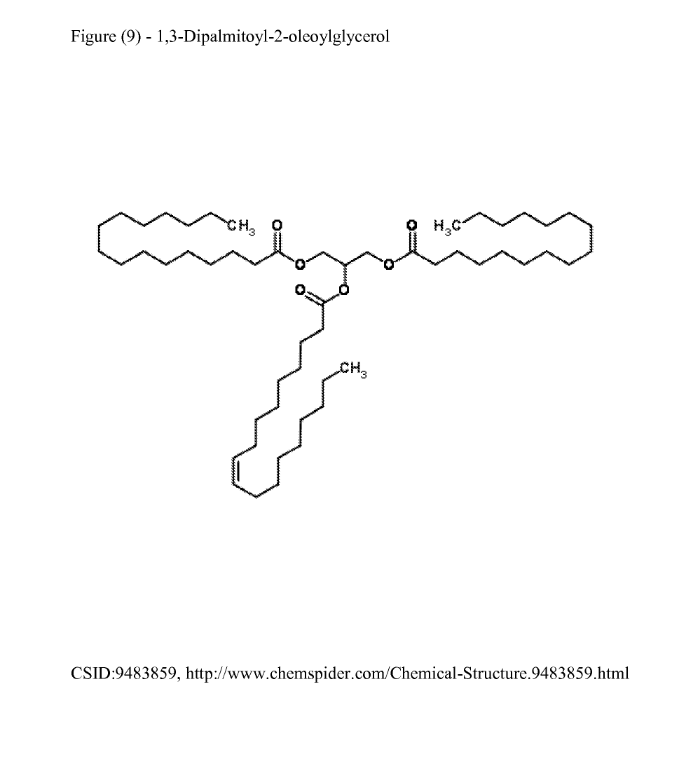

FIG. (10) shows another triglyceride. This particular example shows three identical fatty acids bound to a glycerol carbon by an ester bond. Each of these fatty acids is called Ricinoleic Acid (see FIG. 5) and the common name for the compound is ricinolein.

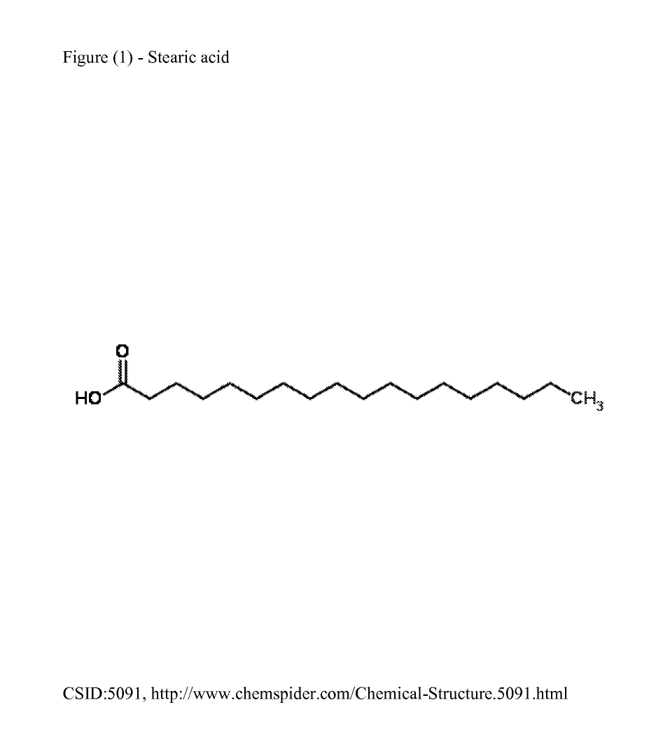

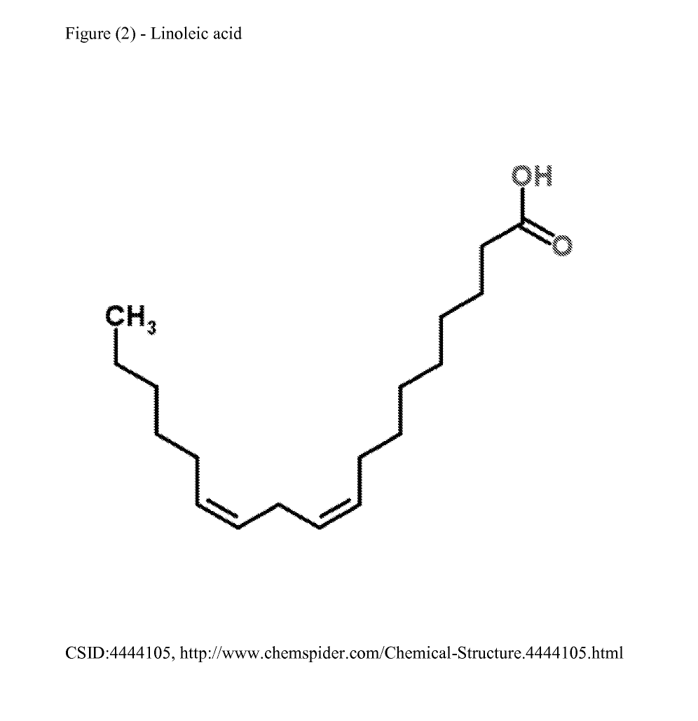

FIG. (11) illustrates a potential mechanism for the trans-esterification of Stearic Acid (see FIG. 1) with Linoleic Acid (FIG. 2) bound up in a triglyceride molecule

FIG. (12) illustrates a potential mechanism for the trans-esterification of Ricinoleic Acid (see FIG. 5) with a generic fatty acid bound up in a triglyceride molecule to produce a ricinoleic ester.

FIG. (13) lists a selection of oils and predominate fatty acid profiles.

### DETAILED DESCRIPTION OF THE INVENTION

The invention is herein described with reference to the aforementioned figures, in which reference numerals refer to like parts throughout. The following description of the invention references specific embodiments in which the invention may be practiced and are intended to describe aspects of the invention in sufficient detail so that one skilled in the art may be successful in reproducing the invention. Other embodiments can be utilized and changes can be made without departing from the scope of the present invention. The present invention is defined by the appended claims and the description should not be taken in a limited sense and shall not limit the scope of equivalents to which such claims are entitled.

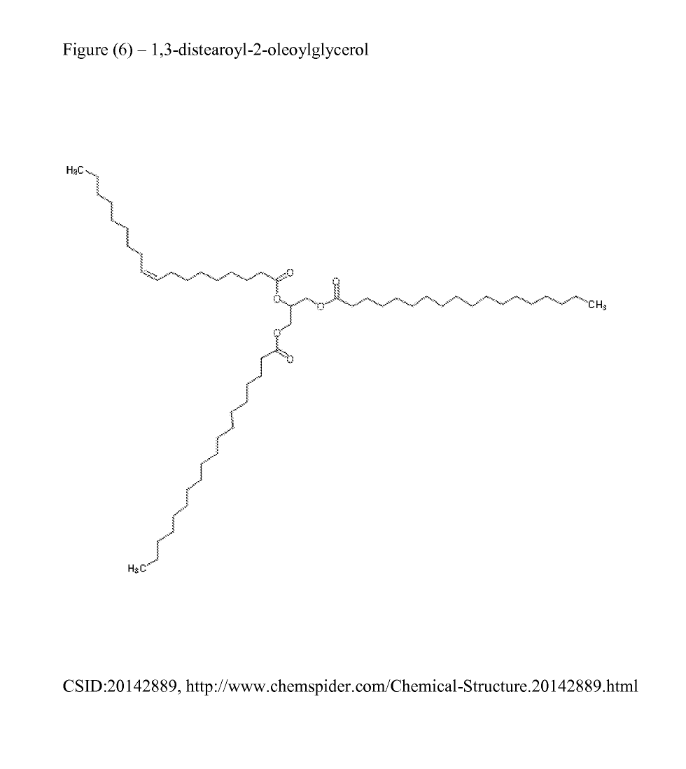

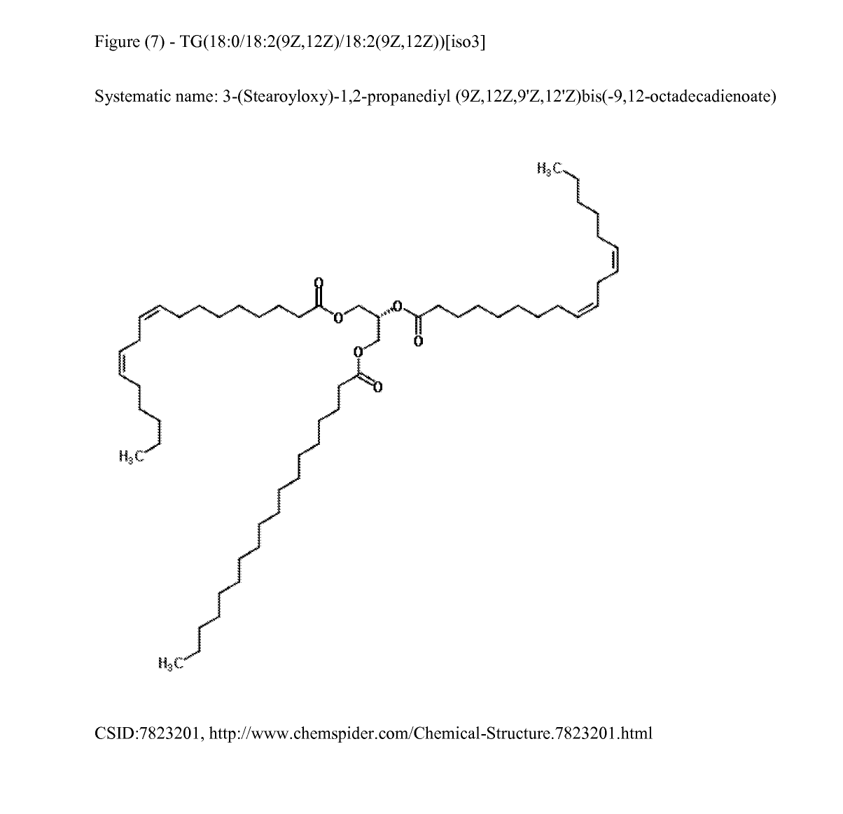

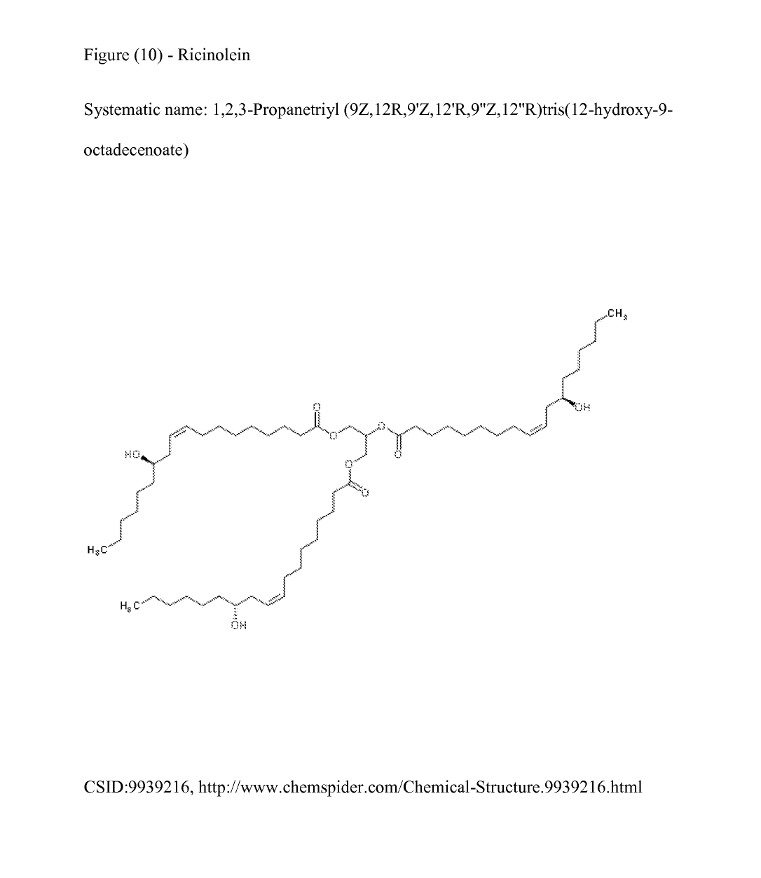

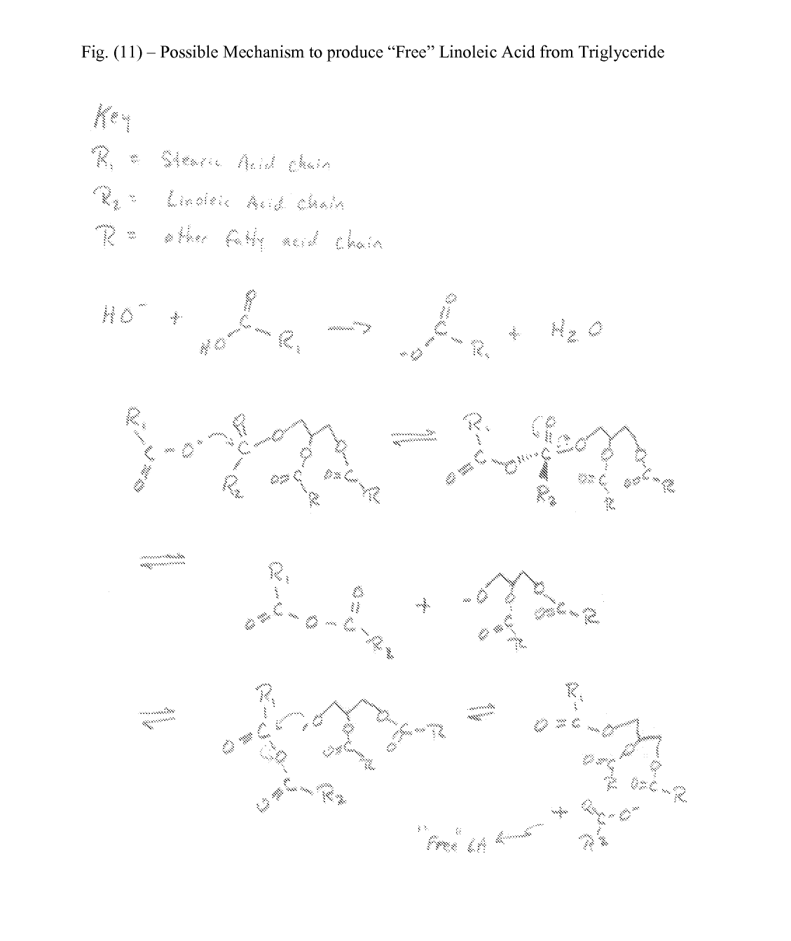

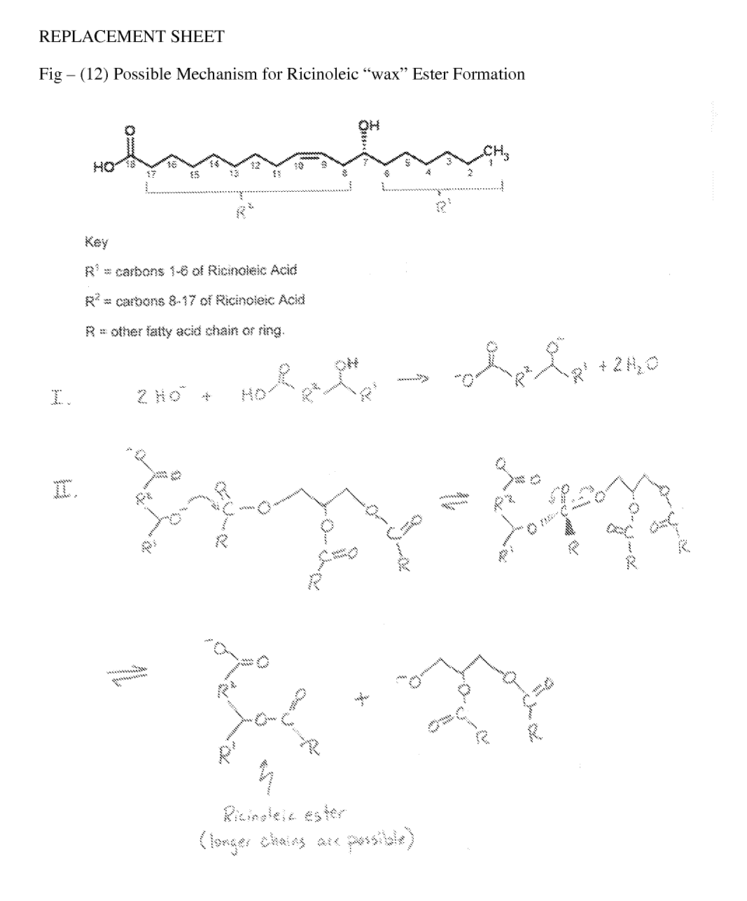

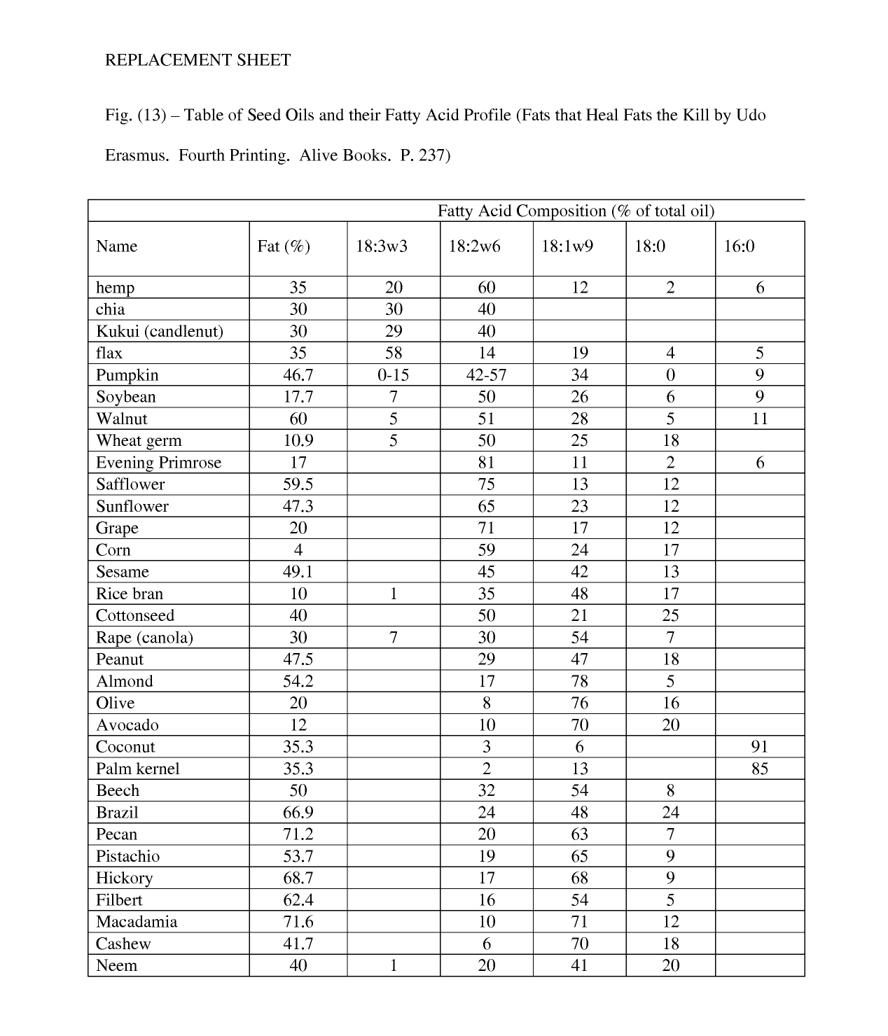

We have only illustrated five examples in FIGS. 6-10, but there are thousands of triglycerides that are comprised of a multitude of fatty acids. In all cases a fatty acid is bound to a glycerol carbon by an ester bond. Triglycerides are a major component in most animal and plant based oils. Some oils have relatively large concentrations of particular fatty acids. For example, olive oil and avocado oil have relatively large percentages of Oleic Acid (FIG. 4) that are bound within the triglyceride structures of these oils. Safflower and sunflower oils contains triglycerides (ex. FIG. 7, FIG. 8 and others) that are comprised of high ratios of Linoleic Acid (FIG. 2), ranging as high as seventy-five percent. Since there are only three fatty acids that can be part of a triglyceride, this would imply that these latter oils have on average at least two of the three available glycerol positions bound with Linoleic Acid and some of these molecules are comprised of Linoleic Acid bound up in all three positions of the glycerol backbone, like the triglyceride illustrated in FIG. 8. Thus these two oils make excellent candidates for emollient compounds that require Linoleic Acid.

Perhaps it is not necessary to state the obvious, but oils, and those containing PUFA in particular, are subject to oxidation. The practitioner should take great care in assuring that the oils used in these procedures are fresh, stored properly, and are not rancid at the outset. It is their very nature that gives PUFA their efficacy in alleviating skin problems. The incorporation of anti-oxidative ingredients, like dl-alpha-tocopheryl, dl-alpha-tocopherol, or vitamin E is prudent.

In the instant invention we chemically cleave a portion of the fatty acids away from their triglyceride structure using a base catalyzed trans-esterification process utilizing a suitable oil (triglyceride) and suitable free fatty acid, like Stearic Acid (SA) although other free fatty acids are conceivable in this process. Some of the fatty acids locked in the triglyceride structure are trans-esterified with the SA and are left to continue the esterification process. A random rearrangement of fatty acids take place in the mixture, but on average a portion of the desired fatty acids will form salts and become freely available in a subsequent acidification step. The relative proportion of free fatty acid salts can be modified by adjusting the concentration of the initial SA. Increasing the concentration of SA will increase the relative proportion of free fatty acid salts. Since there is bound to be trans-esterification reactions taking place, some of the SA takes the position of the target fatty acid locked in the triglyceride structure, leaving the desired fatty acid to be “free.” FIG. 11 illustrates a possible mechanism.

Other novel “wax” esters may also form from the reaction of the Ricinoleic Acids with other fatty acids and triglycerides. FIG. 12 illustrates a potential mechanism for these type reactions.

Inter-Esterification Embodiments

1. Castor Oil.

Castor oil is derived from the castor bean and is a major source of Ricinoleic Acid, which is bound within the triglyceride structure. FIG. (5) shows Ricinoleic Acid. FIG. (10) Illustrates an example of a triglyceride that contains Ricinoleic Acid. This particular fatty acid is unusual in that it contains a hydroxyl group, at the alpha-12 position, which is available for potential esterification reactions via nucleophilic attack (see FIG. 12).

Chemicals Needed:
1. Food grade NaOH (22 g)
2. Distilled water (1200 g)
3. Stearic Acid flakes (30 g)
4. Castor oil (170 g)
5. Food grade citric acid (70 g)

Equipment Needed:
1. 1-L capacity Pyrex measuring cup.
2. ½-L capacity Pyrex measuring cup.
3. High speed stick mixer (KitchenAid model KHB1231ER0)
4. 2000 g capacity scale with 0.1 g precision
5. Digital thermometer
6. Stainless steel spoon
7. Stainless steel knife
8. Fine mesh stainless steel screen or sieve (12.25 cm diameter, 8.9 cm deep)
9. Warming plate
10. Stainless steel flat bowl (at least 2-L capacity)

Procedure:

For these procedures, don appropriate personal protective equipment: lab coat, safety goggles, and neoprene gloves. Work in a well-ventilated hood.

In a well-ventilated area, prepare a 25% NaOH solution by adding 22 grams of NaOH to 66 grams of distilled water contained in a ½-L Pyrex measuring cup or equivalent suitable container. Stir this solution with the stainless steel spoon until the NaOH is fully dissolved. Set aside in a protected and well-ventilated area and allow to cool.

Step One: Inter-Esterification.

Using the 1-L capacity Pyrex measuring cup as its container, prepare a 15% mixture of Stearic Acid in Castor oil. Warm the mixture gently on a hot plate and stir until the Stearic Acid is fully dissolved in solution. The total weight of the solution should be approximately 200 g (30 g SA and 170 g Castor oil). Allow to cool to 46 degrees Celsius. If the cloud point of the mixture is observed, then warm the solution back up to dissolve the crystals and target for a slightly higher fallback temperature. When the target temperature is reached, add the entirety of the 25% NaOH solution to the oil mixture. There should be an immediate reaction as the trans-esterification reaction proceeds. The compound will become very stiff, but continue to manipulate so as to uniformly distribute any excess base. You should observe a corresponding temperature increase of approximately 10-12 degrees Celsius. Manipulate the stiff compound gently using the stainless steel spoon. Within minutes, an aqueous-basic condensate will condense from the stiff compound: decant the base away from the compound and continue to mix gently. When no additional condensate is seen and the compound is fully mixed, cover the container and allow cooling for several hours. You are looking for the temperature to drop by approximately 35 degrees Celsius (or to room temperature). The compound may also be stored over longer time periods if needed.

Step Two: Acidification.

Prepare 700 g of a ten percent solution of citric acid and distilled water by adding 70 g food grade citric acid to 630 grams of distilled water. Set aside the acid solution. Place the compound prepared in Step one into 500 g of distilled water contained within the stainless steel flat bottomed bowl. Using a stainless steel knife, you should cut up the compound into smaller pieces. Once this is accomplished, use the hand blender on high speed to fully homogenize the compound with the distilled water. The pH of this mixture will be very basic: near 14. Continue using the hand mixer while titrating with small amounts of the citric acid solution. Add enough of the citric acid solution until the pH is between 6.5-7.0. At this point, a fatty layer will develop on top of the water and the titration is complete. Separate the fatty layer from the aqueous by spooning the fatty layer onto a fine screen or sieve. Use a stainless steel spoon to manipulate the fatty layer so that all excess water has coalesced and allowed to drain through the screen. The final product is a rich créme, containing ricinoleic fatty acids, that has an emollient effect when applied directly to skin.

2. Other Oils.

We have also demonstrated that the above procedure works with other oils including: apricot oil, avocado oil, safflower oil, grape seed oil, borage oil, evening primrose oil, hemp oil, linseed oil, and jojoba oil (although the properties were markedly different). One important characteristic is smell. It would seem that oils that contained larger amounts of w-3 fatty acids, like linolenic acid, had a slight “fishy” odor when applied to skin. Interestingly, the w-3 fatty acids are a large component of fish oils, so perhaps our observation shouldn't be surprising. This undesirable smell was seen with compounds produced from hemp and linseed oils. Additionally, a compound produced from linseed oil oxidized fairly rapidly and unacceptably. A compound produced from jojoba oil, a so called wax-ester, seemed to be “oily” compared to the others. If one is interested in utilizing other oils with the procedure described in the instant invention, it would be beneficial to prepare each separately and measure the desired effects or quality characteristics prior to their incorporation into a final formulation.

3. Mixtures Combining Individual Inter-Esterified Oils (Prior to the Acidification Step).

One advantage of preparing individual oils as described in this section is that the practitioner may exercise a degree of control in the type and quantity of fatty acids that are cleaved from their triglyceride structure. One can find published charts (FIG. 13) that show the relative proportions of the common fatty acids in most seed or animal based oils. That would mean that if the practitioner desired to have a preponderance of a particular group of fatty acids, they could specifically tailor a compound by choosing specific oil. For example, safflower and sunflower oils contains large quantities of Linoleic Acid as seen in FIG. 13. These would be good candidate oils if the objective is to provide unbound Linoleic Acid in an emollient or skin care compound. FIG. 13 lists a selection of oils along with their predominate fatty acids.

We have found that in concentrations ranging from ten (10) to twenty-five (25) percent SA one can control the relative proportion of unbound fatty acids by increasing the initial SA concentration. By combining the inter-esterified products in varying proportions the practitioner can control the relative proportions of fatty acids in a skin care compound. The following procedure illustrates this idea. We demonstrate the procedure using three inter-esterified oils, but the procedure can be extended to include any number of oils, that have been previously inter-esterified, in various concentrations.

List of Reagents/Compounds:
1. Inter-Esterified Compound of Safflower Oil [20% SA, 80% Safflower oil] (45 g, comprising 45% of the total)
2. Inter-esterified compound of Castor oil [10% SA, 90% Castor oil] (35 g, comprising 35% of the total)
3. Inter-esterified compound of Borage oil [10% SA, 90% Borage oil] (20 g, comprising 20% of the total)
4. Distilled water (860 g)
5. Food grade citric acid (40 g)

Equipment Needed:
1. 1-L capacity Pyrex measuring cup.
2. ½-L capacity Pyrex measuring cup.
3. High speed stick mixer (KitchenAid model KHB1231ER0) or equivalent
4. 2000 g capacity scale with 0.1 g precision
5. Digital thermometer
6. Stainless steel spoon
7. Stainless steel knife
8. Fine mesh stainless steel screen or sieve (12.25 cm diameter, 8.9 cm deep)
9. Warming plate
10. Stainless steel flat bowl (at least 2-L capacity)

Procedure:

For these procedures, don appropriate personal protective equipment: lab coat, safety goggles, and neoprene gloves. Work in a well-ventilated hood.

Step One: Inter-Esterification.

The inter-esterification step has already been completed. You should have prepared the following for this procedure:
1. Inter-esterified compound of Safflower oil [20% SA, 80% Safflower oil]
2. Inter-esterified compound of Castor oil [10% SA, 90% Castor oil]
3. Inter-esterified compound of Borage oil [10% SA, 90% Borage oil]

Step Two: Acidification.

Prepare 400 g of a ten percent solution of citric acid in distilled water and set aside (40 g citric acid added to 360 g distilled water). Weight out: a) 45 g of inter-esterified safflower compound, b) 35 g of inter-esterified castor oil compound, and c) 20 g of inter-esterified Borage oil compound. Place each of the inter-esterified compounds (Castor, Safflower, and Borage), into 500 g of distilled water contained within the flat bottomed stainless steel bowl. Using a stainless steel knife, you should cut up the inter-esterified compounds into smaller pieces. Once this is accomplished, use the hand blender on high speed to fully homogenize the compounds with the distilled water. The pH of this mixture will be very basic: near 14. Continue using the hand mixer while titrating with small amounts of the citric acid solution. Add enough of the citric acid solution until the pH is between 6.5-7.0. At this point, a fatty layer will develop on top of the water and the titration is complete. Separate the fatty layer from the aqueous by spooning the fatty layer onto a fine screen or sieve. Use a stainless steel spoon to manipulate the fatty layer so that all excess water has coalesced and allowed to drain through the screen. The final product is a rich créme, containing LA, GLA, and RA, that has an emollient effect when applied directly to skin.

Intra-Esterification Embodiments

It is possible to forgo the independent preparation of specific oils in the inter-esterification step. The advantage is time savings. The disadvantage would be a loss of some control over the particular fatty acids that are broken free of their triglyceride structure. The following two procedures illustrate possible embodiments.

### Example #1

### Chemicals Needed

1. Food grade NaOH (18 g)
2. Distilled water (914 g)
3. Stearic Acid flakes (28.4 g)
4. Safflower oil (56.8 g)
5. Castor oil (28.4 g)
6. Almond oil (14.2 g)
7. Avocado oil (14.2 g)
8. Food grade citric acid (40 g)

Equipment Needed:
1. 1-L capacity Pyrex measuring cup.
2. ½-L capacity Pyrex measuring cup.
3. High speed stick mixer (KitchenAid model KHB1231ER0)
4. 2000 g capacity scale with 0.1 g precision
5. Digital thermometer
6. Stainless steel spoon
7. Stainless steel knife
8. Fine mesh stainless steel screen or sieve (12.25 cm diameter, 8.9 cm deep)
9. Warming plate
10. Stainless steel flat bowl (at least 2-L capacity)

Procedure:

For these procedures, don appropriate personal protective equipment: lab coat, safety goggles, and neoprene gloves. Work in a well-ventilated hood.

Step One: Intra-Esterification.

In a well-ventilated area, prepare a 25% NaOH solution by adding 18 grams of NaOH to 54 grams of distilled water contained in the ½-L Pyrex measuring cup. Stir this solution with the stainless steel spoon until the NaOH is fully dissolved. Set aside in a protected area and allow to cool.

To the 1-L Capacity Pyrex Container, Add:

1. 56.8 g Safflower oil
2. 28.4 g Castor oil
3. 14.2 g Almond oil
4. 14.2 g Avocado oil
5. 28.4 g Stearic acid

Warm the oil mixture gently on the warming plate and stir until the Stearic Acid is fully dissolved in solution: target temperature for this operation is approximately 60 degrees Celsius. The total weight of the solution should be approximately 142 g. Allow to cool to 46 degrees Celsius. If the cloud point of the mixture is observed, then warm the solution back up to dissolve the crystals and target for a slightly higher temperature. When the target temperature is reached, add the entirety of the 25% NaOH solution to the oil mixture. There should be an immediate reaction as the trans-esterification reaction proceeds. The compound will become very stiff, but continue to manipulate so as to uniformly distribute any excess base. You should observe a corresponding temperature increase of approximately 10-12 degrees Celsius. Manipulate the stiff compound gently using the stainless steel spoon. Within minutes, an aqueous-basic condensate will condense from the stiff compound: decant the base away from the compound and continue to mix gently. When no additional condensate is seen and the compound is fully mixed, cover the container and allow cooling for several hours. You are looking for the temperature to drop by approximately 35 degrees Celsius (or to room temperature). The compound may also be stored over longer time periods if needed.

Step Two: Acidification.

Prepare 400 g of a ten percent solution of citric acid and distilled water (40 g citric acid added to 360 g distilled water). Set aside the acid solution. Place the compound prepared in step one into 500 g of distilled water. Using a stainless steel knife, you should cut up the compound into smaller pieces. Once this is accomplished, use the hand blender on high speed to fully homogenize the compound with the distilled water. The pH of this mixture will be very basic: near 14. Continue using the hand mixer while titrating with small amounts of the citric acid solution. Add enough of the citric acid solution until the pH is between 6.5-7.0. At this point, a fatty layer will develop on top of the water and the titration is complete. Separate the fatty layer from the aqueous by spooning the fatty layer onto a fine screen or sieve. Use a stainless steel spoon to manipulate the fatty layer so that all excess water has coalesced and allowed to drain through the screen. The final product is a rich créme, containing LA, OA, and RA, that has an emollient effect when applied directly to skin.

### Example #2

In this embodiment we utilize an oil that contains 20% dl-alpha-tocopheryl and dl-alpha-tocopherol (vitamin E), which provides an antioxidative effect.

Chemicals Needed:
1. Food grade NaOH (130 g)
2. Distilled water (approx. 5 L)
3. Stearic Acid flakes (200 g)
4. Safflower oil/vitamin E blend (350 g)
5. Castor oil (300 g)
6. Olive oil (100 g)
7. Borage oil (50 g)
8. Food grade citric acid (100 g)

Equipment Needed:
1. 3.5-L capacity Polypropylene container.
2. 1-L capacity Pyrex measuring cup.
3. High speed stick mixer (KitchenAid model KHB1231ER0)
4. 2000 g capacity scale with 0.1 g precision
5. Digital thermometer
6. Large stainless steel spoon
7. Stainless steel knife
8. Fine mesh stainless steel screen or sieve (12.25 cm diameter, 8.9 cm deep)
9. Stainless steel flat bottom container (10-L capacity).
10. Microwave oven.

Procedure:

For these procedures, don appropriate personal protective equipment: lab coat, safety goggles, and neoprene gloves. Work in a well-ventilated hood.

Step One: Intra-Esterification.

In a well-ventilated area, prepare a 25% NaOH solution by adding 130 grams of NaOH to 390 grams of distilled water contained in the 1-L Pyrex measuring cup. Stir this solution with the stainless steel spoon until the NaOH is fully dissolved. Set aside in a protected area and allow to cool.

To the 3.5-L capacity Polypropylene container, add:
1. 350 g Safflower oil/vitamin E blend
2. 300 g Castor oil
3. 100 g Olive oil
4. 50 g Borage oil
5. 200 g Stearic acid

Place the 3.5-L polypropylene container with the oils and stearic acid into a microwave oven. Heat for two minutes. The target temperature for this operation is approximately 60 Celsius. Using the stainless steel spoon, stir until the Stearic Acid is fully dissolved in solution. The total weight of the solution should be approximately 1000 g. Allow to cool to 46 degrees Celsius. If the cloud point of the mixture is observed, then warm the solution back up to dissolve the crystals and target for a slightly higher fallback temperature, then allow cooling to 46 degrees Celsius. When the target temperature is reached, add the entirety of the 25% NaOH solution to the oil mixture. There should be an immediate reaction as the trans-esterification reaction proceeds. The compound will become very stiff, but continue to manipulate so as to uniformly distribute any excess base. You should observe a corresponding temperature increase of approximately 10-12 degrees Celsius. Manipulate the stiff compound gently using the stainless steel spoon. Within minutes, an aqueous-basic condensate will condense from the stiff compound: decant the base away from the compound and continue to mix gently. When no additional condensate is seen and the compound is fully mixed, cover the container and allow cooling for several hours. You are looking for the temperature to drop by approximately 35 degrees Celsius (or to room temperature). The compound may also be stored over longer time periods if needed.

Step Two: Acidification.

Prepare 1000 g of a ten percent solution of citric acid and distilled water (by adding 100 g citric acid to 900 g distilled water). Set aside the acid solution. Place the compound prepared in step one into 4 Liters of distilled water contained in a large stainless steel vessel. Using a stainless steel knife, you should cut up the intra-esterified compound into small pieces. Once this is accomplished, use the hand blender on high speed to fully homogenize the compound with the distilled water. The pH of this mixture will be very basic: near 14. Continue using the hand mixer while titrating with small amounts of the citric acid solution. If desired, a fragrance may be added when the pH is near 8.0. Add enough of the citric acid solution until the pH is between 6.5-7.0. At this point, a fatty layer will develop on top of the water and the titration is complete. Separate the fatty layer from the aqueous by spooning the fatty layer onto a fine screen or sieve. Use a stainless steel spoon to manipulate the fatty layer so that all excess water has coalesced and allowed to drain through the screen. The final product is a rich créme that has an emollient effect when applied directly to skin.

Other Variants of the Above Procedure

There are many other variants possible utilizing other oils in various ratios. We have successfully prepared emollient crémes using subsets from the following: Abyssinian oil, avocado oil, apricot oil, borage oil, castor oil, evening primrose oil, hemp oil, jojoba oil, linseed oil, meadowfoam oil, oleic acid, olive oil, pomegranate oil, safflower oil, stearic acid, and sunflower oil. Other oils may be chosen depending on the fatty acid content desired.

Proper Storage and Packaging

As previously mentioned, the unsaturated fatty acids and the PUFA in particular are subject to oxidation. It is very important to properly store and pack these emollient compounds once they are produced. These compounds should not be exposed to strong light or any heat sources. Ideally they should be kept between 7 and 24 degree Celsius and certainly not continuously exposed to sunlight or intense light sources. Packaging is best in airless bottles or like containers so as to minimize their contact with oxygen. It may be prudent to utilize oxygen-free atmospheres like nitrogen in purging packaging containers.

If delay is expected between production and packaging steps it is prudent to store these emollient compounds either in vacuum packed bags, containers or like devices to minimize oxygen contact. Longer term storage may be accomplished in refrigerated environments.

The descriptions described above and illustrated in the drawings are presented by way of example only and are not intended to limit the concepts and principles of the instant invention. Thus, we have described several embodiments of a novel invention. As is evident from the foregoing discussion and description, certain aspects of the present invention are not limited by the particular details of the examples illustrated herein, and it is likely that other modifications, applications, or equivalents thereof, will occur to those skilled in the art. The terms “having,” “including,” “like” and similar terms as used in the foregoing specification are used in the sense of “may include” and are not necessarily “required”. There are many options, methods and reagents that can accomplish the required chemical reactions and changes in physical state described in the present formulations, that may become apparent to those skilled in the art after considering the overall process, figures, and chemical processes described herein. All such variants, modifications, other uses and applications which do not depart from the spirit and scope of the invention are deemed to be covered by the invention which is limited only by the claims which follow.

1. A lipid compound derived from blends of dl-alpha-tocopheryl, dl-alpha-tocopherol, a free fatty acid, and oils of varying concentrations containing the polyunsaturated fatty acids, linoleic acid, gamma-linolenic acid, ricinoleic acid, and oleic acid that are made freely available for use in skin formulations.

2. The formulation of claim (1) that has been saponified.

3. The formulation of any one of the claims (1, 2) that has been acidified.

4. The formulation of any one of the claims (1, 2, 3) that has been compounded with other substances to produce: soaps, cosmetics, lip balms, pharmaceuticals, emollients, skin creams, lotions.

5. The formulation of any one of the claims (1, 2, 3, 4) that contains free fatty acids or fatty acid salts.

6. The formulation of any one of the claims (1, 2, 3, 4) that may be incorporated with other compounds, including oils, water, cosmetic formulations, or pharmaceutical formulations.

7. The formulation of claim (1) further comprising an additional component selected from additional oils, individual fatty acids and combinations thereof.

8. The formulation of claim (7), wherein the additional component is an additional oil.

9. The formulation of claim (8), wherein the additional oil is selected from almond oil, avocado oil, black current oil, borage oil, castor oil, chia seed oil, chullu seed oil, coconut oil, corn oil, emu oil, evening primrose oil, flax seed oil, genetically modified corn oil, grape seed oil, hemp seed oil, jojoba seed oil, kamala seed oil, kapok oil, karanja seed oil, mustard seed oil, (niger seed oil, olive oil, palash oil, palm oil, pecan oil, perilla oil, punna oil, red currant oil, rice bran oil, rubber seed oil, safflower oil, sesame oil, sorghum oil, soya bean oil, sunflower oil, thumba seed oil, tobacco seed oil, walnut oil, wheat germ oil, and mixtures thereof.

10. The formulation of claim (8), wherein the additional oil is obtained from a genetically modified organism, like bacteria, blue green algae, plants or animals.

11. The formulation of claim (7), wherein the additional component is an individual fatty acid.

12. The formulation of claim (11), wherein the additional fatty acid is stearic acid.

13. The formulation of claim (11), wherein the additional fatty acid is obtained from a genetically modified organism, like bacteria, blue green algae, plants or animals.

14. The formulation of claim (8), wherein the additional oil is obtained from animal sources.

15. The formulation of claim (11), wherein the additional fatty acid is obtained from animal sources.

16. The formulation of any one of the claims (11, 12, 13) wherein the additional fatty acid initiates or catalyzes a trans-esterification, inter-esterification, or intra-esterification reaction.

17. The formulation of any one of the claims (1, 2, 3, 4, 5, 6, 7) further comprising oils that are trans-esterified.

18. The formulation of any one of the claims (1, 2, 3, 4, 5, 6, 7) further comprising oils that are intra-esterified.

19. The formulation of any one of the claims (1, 2, 3, 4, 5, 6, 7) that contains ricinoleic esters.

20. The formulation of any one of the claims (1, 2, 3, 4, 5, 6, 7) that provides an emollient effect to the skin.

21. The formulation of any one of the claims (1, 2, 3, 4, 5, 6, 7) that provides the essential fatty acid, linoleic acid in free form.

22. The formulation of any one of the claims (1, 2, 3, 4, 5, 6, 7) that is packaged in an airless container, package or bottle.

23. The formulation of any one of the claims (1, 2, 3, 4, 5, 6, 7) that is de-aerated.

24. The formulation of any one of the claims (1, 2, 3, 4, 5, 6, 7) that is deodorized.

25. The formulation of any one of the claims (1, 2, 3, 4, 5, 6, 7) that contains anti-oxidants.
